(19) United States
(12) Patent Application Publication
(10) Pub. No.: US 2017/0332922 A1
(43) Pub. Date: Nov. 23, 2017

(54) STROKE DETECTION USING OCULAR PULSE ESTIMATION

(71) Applicant: Welch Allyn, Inc., Skaneateles Falls, NY (US)

(72) Inventor: David E. Quinn, Auburn, NY (US)

(21) Appl. No.: 15/597,801

(22) Filed: May 17, 2017

Related U.S. Application Data
(60) Provisional application No. 62/338,150, filed on May 18, 2016.

(51) Int. Cl. A61B 5/022; A61B 3/14; A61B 3/00; A61B 3/16; A61B 8/10; A61B 5/00

(52) U.S. Cl. CPC A61B 5/02216; A61B 8/10; A61B 5/7275; A61B 5/6803; A61B 5/0075; A61B 3/0033; A61B 3/165; A61B 3/14; A61B 3/0041; A61B 5/0022

(57) ABSTRACT

A system and method for detecting a stroke includes a non-contact ocular pulse measurement device configured to output a first and a second ocular pulse measurement signals for each of a patient's eyes, respectively. A computing system has a processor and a memory, and the memory stores instructions that when executed cause the processor to analyze the first and second ocular pulse measurements. An index of difference between the first and second ocular pulse measurements is determined, and a user interface is generated that includes a stroke advisory to the patient based on the index of difference.

### CROSS REFERENCE TO RELATED APPLICATIONS

This applications claims the benefit of U.S. Provisional Application No. 62/338,150, filed May 18, 2016, and titled “Stroke Detection Using Ocular Pulse Estimation,” the disclosure of which is hereby incorporated herein by reference.

### INTRODUCTION

Stroke is a serious and often deadly condition that happens rapidly and often without a detectable precursor. Although there are known risk factors such as high blood pressure and high cholesterol, often there is no warning sign detected before a stroke occurs. Signs and symptoms of a stroke may include an inability to move or feel on one side of the body, problems understanding or speaking, loss of vision to one side among others. However, signs and symptoms such as these typically appear after the stroke has occurred. Early recognition of stroke is deemed important as this can expedite diagnostic tests and treatments. A major stroke is often preceded by transient ischemic attacks (TIA) or ministrokes. Early detection of evidence of the types of events could alert the patient that intervention is needed before a major stroke occurs.

### SUMMARY

In one aspect, an apparatus for detecting a stroke includes a non-contact ocular pulse measurement device configured to output a first and a second ocular pulse measurement signals for each of a patient's eyes, respectively. A computing system has a processor and a memory, and the memory stores instructions that when executed cause the processor to analyze the first and second ocular pulse measurements. An index of difference between the first and second ocular pulse measurements is determined, and a user interface is generated that includes a stroke advisory to the patient based on the index of difference.

In another aspect, a method for detecting a stroke includes measuring an ocular pulse for two eyes of a patient using a non-contact measurement. The ocular pulse measurements are compared between the two eyes, and a user interface is generated that includes a stroke advisory to the patient based on the comparison.

### DESCRIPTION OF THE FIGURES

The following figures, which form a part of this application, are illustrative of described technology and are not meant to limit the scope of the claims in any manner, which scope shall be based on the claims appended hereto.

FIG. 1 is a flow diagram illustrating aspects of an example of a method for detecting a stroke.

FIG. 2 is a block diagram illustrating an example of a system for detecting a stroke.

FIG. 3 illustrates an example of a wearable optical pulse measurement system mounted in a pair of eyeglasses.

FIG. 4 is a block diagram illustrating another example of a system for detecting a stroke.

FIG. 5 is a block diagram illustrating aspects of an example computing system suitable for use in the systems shown in FIGS. 2 and 3.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the spirit or scope of the present disclosure. The following detailed description is therefore not to be taken in a limiting sense.

This disclosure relates generally to methods and systems that use an ocular pulse measurement to detect carotid artery stenosis (CAS), a condition that clogs or blocks the arteries that feed the front part of the brain. A patient may then be advised of the possibility of stroke based on the detection of CAS. Thus, in some implementations, a system is provided that provides early indication of impending stroke, allowing a patient to seek medical attention before the onset of a stroke.

In some embodiments, an ocular pulse amplitude (OPA) estimation is made using a non-contact measurement device. For example, an eyeglass mounted device and a distance measuring technology to detect the amplitude of eye pulsations due to heartbeats may be employed. Examples of distance measurement technologies include digital image of the pulsating eye, infrared (IR) distance measurement to the cornea, ultrasonic time of flight measurement to the cornea, and arc of the eye changes using directed light.

Generally, if there is restriction of blood flow on one side of the brain, one eye will pulsate with an amplitude different than the other eye. In normal situation, both eyes have same pulse amplitude, phase and pulse morphology.

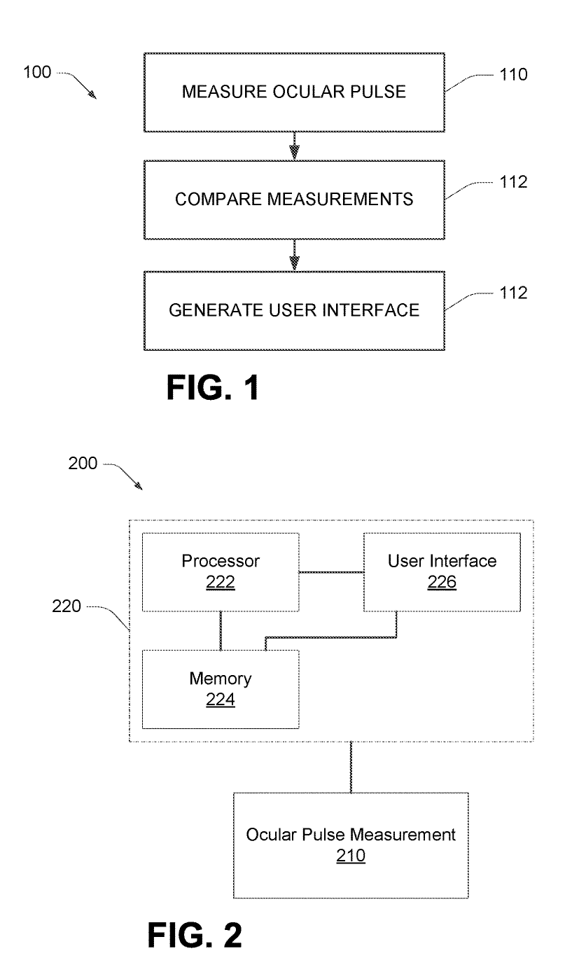

FIG. 1 illustrates a method 100 for detecting a stroke in a patient. As shown in block 110, an ocular pulse is measured for both eyes of a patient. The measured ocular pulse is compared between the patient's two eyes, and a user interface is generated that includes a stroke advisory to the patient based on the comparison.

FIG. 2 illustrates an example of a stroke detection system 200 that executes the method shown in FIG. 1. The system 200 includes a non-contact ocular pulse measurement device 210 that outputs ocular pulse measurement signals to a computing device 220. The output signal from the measurement device 210 may include, for example, first and second ocular pulse measurement signals for each of a patient's eyes, respectively. The computing device 220 includes a processor 222 and a memory 224 accessible by the processor. The memory 222 stores program instructions that when executed cause the processor 221 to analyze the first and second ocular pulse measurements, determine an index of difference between the first and second ocular pulse measurements, and generate a user interface 226 including a stroke advisory to the patient based on the index of difference. In other words, if the ocular pulse measurement of one eye differs from the measurement of the other eye by more than some predetermined amount, a stroke warning or advisory may be presented to the patient via the user interface 226. The user interface 226 may be a local component of the computing device 220, or it could be located remotely from the processor 222 and memory 224.

In some embodiments, the ocular pulse measurement is an ocular pulse amplitude (OPA) measurement, in which the natural pulsations (displacement) of the eyes due to pressure pulsations are measured. Known OPA measurement devices typically require contact with the surface of the patient's eyes. However, some example systems disclosed herein use a non-contact OPA measurement, making it easier for patients to take measurements outside of a clinic setting. Providing a system for use “at home” may increase the chances of early stroke detection and treatment. Examples of OPA measurement devices include the Diaton Tonometer from Bicom, Inc. of Long Beach, N.Y. (http://tonometer-diaton.com/?gclid=CPDVsv-A08wCFYpZhgodIJ0HzQ).

For instance, in some implementations the OPA comparison includes comparing the measured ocular pulse in a time domain. A particular point, such as the start of the pulse, on the OPA measurement wave form for one eye may be compared to the same point on the other eye. If this time point varies between the patient's two eyes by more than some threshold, it may indicate a stroke condition. In other embodiments, an amplitude domain comparison is used in place of, or in addition to, the time domain comparison. For example, the height of the ocular pulse may be compared between the patient's eyes, and if the difference in pulse amplitude varies by more than some threshold, it may indicate a stroke condition. Comparisons may also be made in the frequency domain to detect significant ocular pulse shape differences.

The OPA comparison may also include comparing the measured ocular pulse to a baseline ocular pulse measurement. For example, previous measurements for a patient could be stored in the memory 224, or another memory. The real time OPA measurement may then be compared to the previous measurements and used to determine a risk of stroke for the patient.

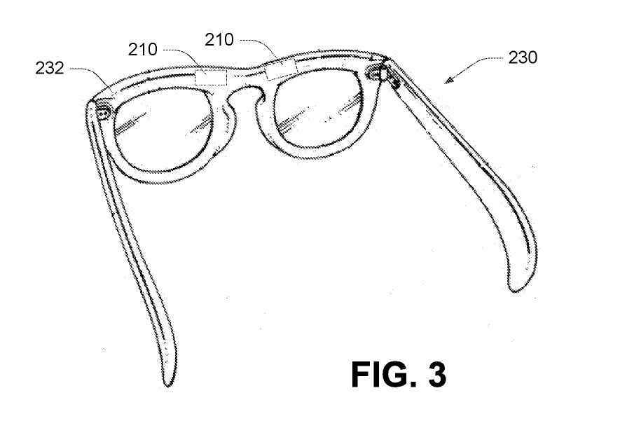

In some example systems, the ocular pulse measurement device 210 is incorporated into a wearable device, such as eyeglasses worn by the patient. Further, since the measurement device 210 is frequently situated near the patient's eyes, the system 200 may be programmed to periodically perform the OPA measurement and comparison process. FIG. 3 illustrates an example in which one or more ocular pulse measurement devices 210 are incorporated into the frame 232 of a pair of eyeglasses 230.

Some OPA measurement techniques that may be employed in various embodiments include making digital images of the patient's eyes, making infrared (IR) distance measurements to corneas of the patient's eyes, making ultrasonic time of flight measurements to the patient's corneas, and measuring the arcs of the patient's eyes using a directed light. Some example OPA measurement devices 210 further provide a focus point for the patient. Focusing on an object or point reduces the patient's eye movements, reducing changes in the measured OPA due to moving eyes.

In some examples, the OPA measurement device 210 and computing device 220 are integrated into a single piece of equipment for use by a patient. In other examples, the measurement device 210 is a separate component that provides the measurement signals to a computing device 220 located remotely from the measurement device 210. The computing device 220 could be implemented, for example, by any appropriately-programmed computing device such as a desktop computer, a laptop computer, a mobile telephone, a smart phone, a tablet personal computer, etc. The output signals from the OPA measurement device 210 may be provided to the computing device 220 by any suitable means, including wired and/or wireless transmissions.

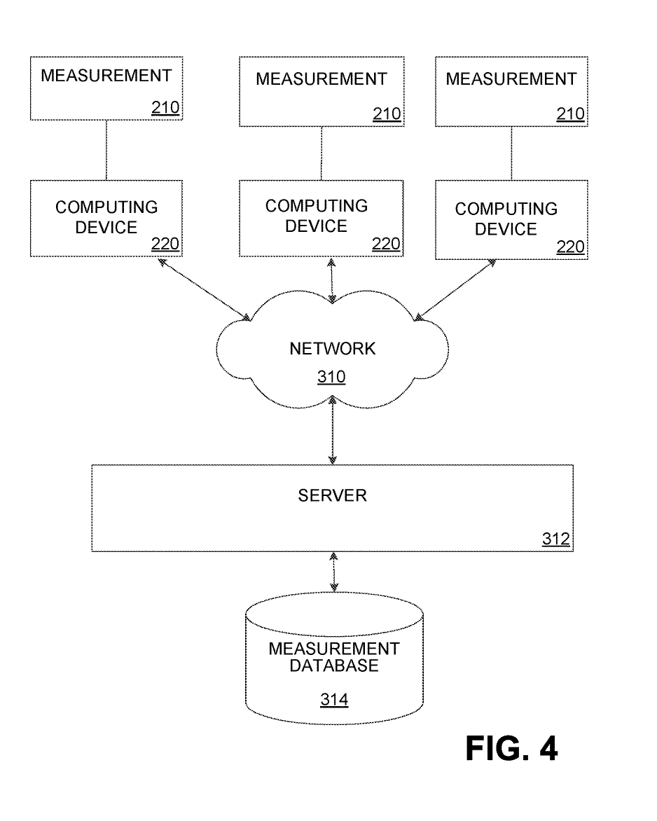

FIG. 4 illustrates a system 300 in which several OPA measurement devices 210 output measurement signals to a central computing device, such as a server computer 312. The measurement signals may be stored in a central database 314. Signal analysis may be performed by the server computer 312 in place of, or in addition to measurement signal analysis by a computing device 220 local to the patient. For example, a computing device such as the patient's smart phone may OPA measurement receive signals from the measurement device 210 and relay the raw signals to the server 312 and database 314 via a network 310 such as the internet. The user interface 226 could then be generated by the server computer 312 and transmitted to the computing device 220 and/or other devices for the patient. For example, a stroke advisory message could be generated by the server computer 312 based on the OPA measurement analysis, then sent to the patent via the network 310 in the form of an email or text message. In other implementations, signal processing is done by the local device 220, and this information may be transmitted to the server 312 and database 314 for further analysis, baseline comparisons, archival, etc.

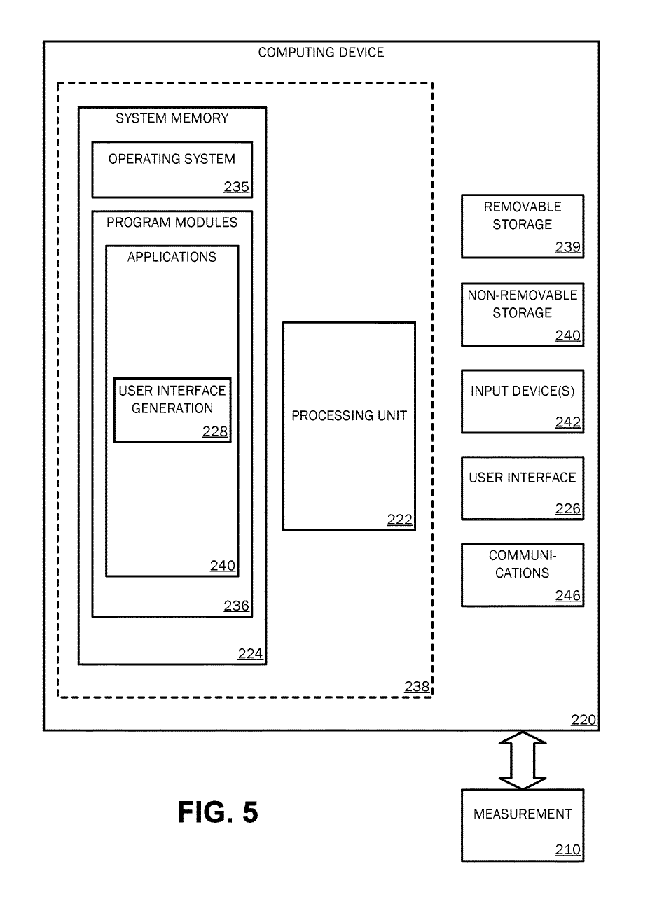

FIG. 5 is a block diagram illustrating physical components (i.e., hardware) of a computing device 220 with which embodiments of the disclosure may be practiced. The computing device components described below may be suitable to act as the computing devices described above, such as the computing devices illustrated in FIGS. 2 and 3 discussed above. In a basic configuration, the computing device 220 may include at least one processor 222 and a system memory 224. Depending on the configuration and type of computing device, the system memory 224 may comprise, but is not limited to, volatile storage (e.g., random access memory), non-volatile storage (e.g., read-only memory), flash memory, or any combination of such memories. The system memory 224 may include an operating system 235 and one or more program modules 236 suitable for running software applications 238. The operating system 235, for example, may be suitable for controlling the operation of the computing device 220. Furthermore, embodiments of the disclosure may be practiced in conjunction with a graphics library, other operating systems, or any other application program and is not limited to any particular application or system. This basic configuration is illustrated in FIG. 5 by those components within a dashed line 238. The computing device 220 may have additional features or functionality. For example, the computing device 220 may also include additional data storage devices (removable and/or non-removable) such as, for example, magnetic disks, optical disks, or tape. Such additional storage is illustrated in FIG. 4 by a removable storage device 239 and a non-removable storage device 240.

As stated above, a number of program modules and data files may be stored in the system memory 224. While executing on the processing unit 222, the program modules 236 may perform processes including, but not limited to, the OPA signal analysis discussed above. Other program modules that may be used in accordance with embodiments of the present disclosure, and in particular a user interface generation module 228 to generate the user interface 226, may include electronic mail and contacts applications, word processing applications, spreadsheet applications, database applications, slide presentation applications, drawing or computer-aided application programs, etc.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 4 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, may be operated via application-specific logic integrated with other components of the computing device 220 on the single integrated circuit (chip). Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

The computing device 220 may also have one or more input device(s) 242 such as a keyboard, a mouse, a pen, a sound or voice input device, a touch or swipe input device, etc. The user interface 226 may include various output components such as a graphics display, speakers, a printer, etc. The aforementioned devices are examples and others may be used. The computing device 220 may include one or more communication connections 246 allowing communications with the OPA measurement device 210 and other computing devices. Examples of suitable communication connections 246 include, but are not limited to, RF transmitter, receiver, and/or transceiver circuitry; universal serial bus (USB), parallel, and/or serial ports.

The term computer readable media as used herein may include non-transitory computer storage media. Computer storage media may include volatile and nonvolatile, removable and non-removable media implemented in any method or technology for storage of information, such as computer readable instructions, data structures, or program modules. The system memory 224, the removable storage device 239, and the non-removable storage device 240 are all computer storage media examples (i.e., memory storage.) Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 220. Any such computer storage media may be part of the computing device 220. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Embodiments of the present invention may be utilized in various distributed computing environments where tasks are performed by remote processing devices that are linked through a communications network in a distributed computing environment.

The block diagrams depicted herein are just examples. There may be many variations to these diagrams described therein without departing from the spirit of the disclosure. For instance, components may be added, deleted or modified.

While embodiments have been described, it will be understood that those skilled in the art, both now and in the future, may make various improvements and enhancements can be made.

The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application. Regardless whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the claimed invention and the general inventive concept embodied in this application that do not depart from the broader scope.

What is claimed is:

1. A system for detecting a stroke, comprising:
a non-contact ocular pulse measurement device configured to output a first and a second ocular pulse measurement signals for each of a patient's eyes, respectively;
a processor and a memory, the memory storing instructions that when executed cause the processor to
analyze the first and second ocular pulse measurements;
determine an index of difference between the first and second ocular pulse measurements; and
generate a user interface including a stroke advisory to the patient based on the index of difference.

2. The system of claim 1, wherein the ocular pulse measurement includes an ocular pulse measurement.

3. The system of claim 1, wherein the instructions cause the processor to:
compare the measured ocular pulse in a time domain.

4. The system of claim 1, wherein the instructions cause the processor to:
compare the measured ocular pulse in at least one of an amplitude domain and a frequency domain.

5. The system of claim 1, wherein the instructions cause the processor to:
compare the measured ocular pulse to a baseline ocular pulse measurement.

6. The system of claim 1, wherein:
the a non-contact ocular pulse measurement device includes an eyeglass mounted measurement device. The system of claim 1, wherein the instructions cause the processor to:
make digital images of the patient's eyes.

8. The system of claim 1, wherein the instructions cause the processor to:
make infrared (IR) distance measurements to corneas of the patient's eyes.

9. The system of claim 1, wherein:
the non-contact ocular pulse measurement device includes a directed light generator; and
wherein the instructions cause the processor to make ultrasonic time of flight measurements to the corneas and the arcs of the patient's eyes using the directed light.

10. The system of claim 1, further comprising:
an output device;
wherein the generated user interface is displayed on the output device.

11. A method for detecting a stroke, comprising:
measuring an ocular pulse for two eyes of a patient using a non-contact measurement;
comparing the measured ocular pulse between the two eyes;
generating a user interface including a stroke advisory to the patient based on the comparison.

12. The method of claim 11, wherein:
measuring the ocular pulse includes measuring an ocular pulse amplitude.

13. The method of claim 11, wherein:
comparing the measured ocular pulse includes comparing the measured ocular pulse in a time domain.

14. The method of claim 11, wherein:
comparing the measured ocular pulse includes comparing the measured ocular pulse in at least one of an amplitude domain and a frequency domain.

15. The method of claim 11, wherein:
comparing the measured ocular pulse includes comparing the measured ocular pulse to a baseline ocular pulse measurement.

16. The method of claim 11, wherein:
measuring the ocular pulse includes using an eyeglass mounted measurement device.

17. The method of claim 11, wherein:
measuring the ocular pulse includes making digital images of the patient's eyes.

18. The method of claim 11, wherein:
measuring the ocular pulse includes making infrared (IR) distance measurements to corneas of the patient's eyes.

19. The method of claim 11, wherein:
measuring the ocular pulse includes making ultrasonic time of flight measurements to the corneas and the arcs of the patient's eyes using a directed light.
